(19) United States
(12) Patent Application Publication
(10) Pub. No.: US 2012/0058072 A1
(43) Pub. Date: Mar. 8, 2012

(54) Compositions for Removal of Odors

(76) Inventors: Alexander Deiser, Boca Raton, FL (US); Claudia Nettig, Boca Raton, FL (US)

(21) Appl. No.: 12/877,500

(22) Filed: Sep. 8, 2010

(51) Int. Cl. A61L 9/01

(52) U.S. Cl. 424/76.1

(57) ABSTRACT

The present invention provides, in one embodiment, a composition for removing odors. The composition includes, in one embodiment, a salt having at least one of a halogen and an alkaline in an effective amount sufficient to remove odors from the materials when applied thereto. The salt, in an embodiment, may include one of chloride, sodium, magnesium, calcium, potassium, or a combination thereof. A method of making the composition is also provided herein.

### TECHNICAL FIELD

The present invention generally relates to odor removal. More particularly, the invention relates to a composition for odor-removal and a method that may be used to remove odors from materials.

### BACKGROUND

Unappealing odors are present in all places of society. For example, materials that are exposed to cooking odor, smoke, and other unappealing odor can often naturally absorb into clothing or furniture and can remain for an extended period of time. Because such odors are objectionable and may make the environment seem unpleasant, various compositions have been developed for reducing odors. Many of these compositions, however, only substitute other odors for foul odors. Aromas or perfumes may be used to mask foul odors. While an aroma added to a foul odor may be inviting to some people, it may be disliked by other people or cause an allergic response. Further, the addition of such an aroma to undesirable odors may create another smell that is more disliked than the original odor.

The control of odor has also involved chemical modification methods. Conventional chemical modification methods may utilize oxidative agents, such as oxygen bleaches, chlorine, sodium hypochlorite, chlorine dioxide, and potassium permanganate to reduce foul odors via oxidative degradation. Reductive degradation which uses reducing agents such as sodium bisulfite has also been employed to reduce malodor. Unfortunately, these types of oxidative agents and reducing agents may damage colored fabrics by bleaching and/or discoloring the colored fabrics. Odor absorption has also become a popular form of reducing odors. Commonly known odor absorbers include activated charcoal and zeolites. Unfortunately, these solids can also be harmful to fabrics. Activated charcoal may stain light colored fabrics, and zeolites may roughen the texture of the fabric if too much is applied to the fabric.

A major problem in today's active lifestyle is the removal of body and sweat odors in our clothing and fabrics, such as high tech fabrics. Even after washing, boiling or treating the clothing or fabrics through chemical dry cleaning or special detergent, the bacteria that causes the odor may still be present. Once the bacteria encounter more sweat, the bacteria can once again activate the smell and odor in clothing and in fabrics.

Accordingly, it would be desirable to have a composition that can destroy the bacteria or remove the source that causes the odor, while minimizing harm to the fabrics, rather then merely masking the odor with a scent.

### SUMMARY OF THE INVENTION

The present invention provides, in an embodiment, a composition for removing odors. The composition includes, in one embodiment, a salt having at least one of a halogen and an alkaline in an effective amount sufficient to remove odors from the materials when applied thereto. The salt, in an embodiment, may include one of chloride, sodium, magnesium, calcium, potassium, or a combination thereof.

The present invention provides, in accordance with another embodiment, a method for making a composition for removing odors. The method includes, in one embodiment, obtaining a salt having at least one of a halogen and an alkaline, and processing the salt, such that it is suitable for administration to a material, and is in an effective amount sufficient to remove odors from materials when applied thereto. The salt, in an embodiment, may include one of chloride, sodium, magnesium, calcium, potassium, or a combination thereof.

### BRIEF DESCRIPTION OF DRAWINGS

FIG. 1 illustrates a diagram of the components in the odor-removing composition.

FIG. 2 illustrates a chart of the components in the odor-removing composition.

FIG. 3 illustrates chemical properties of the components in the odor-removing composition.

### DESCRIPTION OF SPECIFIC EMBODIMENTS

In accordance with an embodiment of the present invention, a composition for removing odors or reducing sources of odors from a material includes a solution of a salt mixture. The salt mixture may include, for instance, salt ions, such as chloride, sodium, sulfate, magnesium, calcium, potassium, or any combination thereof. The salt mixture, in some cases, may also include other constituents, such as, bromine, bicarbonate, borate, strontium, carbon or fluoride. Of course, other constituents may also be used if desired.

Humans sweat, even without physical or sports activity. As the body sweats, sweat bacteria can start growing on the skin and can produce the unpleasant odor which subsequently penetrates and permeates through the fabric. Sweat by itself does not have any odor. However, when the sweat interacts with the skin, bacteria starts to grow, which subsequently can produce an unpleasant smell. Sweat bacteria or related biological organism can typically be oil-based and can act to stick to synthetic fiber and fabrics.

Due to its oil-based nature, it may be difficult or impossible to remove the bacteria from synthetic fibers and fabrics even with the use of various detergents, boiling, or chemical cleaning treatments. When any fabric is washed or cleaned, the resulting washed fabric can smell clean because of the fragrance of the detergent/soap, which generally provides a false sense of cleanliness. However, as soon as the bacterium interacts with new sweat, it may be activated and release a terrible odor until the fabric becomes beyond repair. After repeating the process for several months, synthetics or fabrics may be reaching a point of bacteria infestation. This can lead to infections, as well as dermatological issues.

The odor-removal composition of the present invention may include, in an embodiment, a salt mixture in an amount suitable for administration to various materials, and in an effective amount sufficient to remove odors from the materials. The composition can act to destroy the bacteria that causes the odor rather then just masking the odor. By destroying or eliminating the bacteria, the bacteria are no longer alive or active cannot produce any smell.

In accordance with an embodiment of the present invention, the odor-removal composition can be used to remove the odors from a variety of materials including natural fabrics, such as clothing or furniture; synthetic fabrics, such as clothing or furniture, high tech fabrics, specialty fabrics (e.g., Dry Fit® or Cool Max®); carpeting; counter tops; plastic and metal surfaces (e.g., wrist watch and wrist watch band in plastic and metal, metal jewelry, jewelry clasp, etc.); synthetic and leather goods; tile floors; humans, such as armpits, foot, hair and body; as well as pets. The odor-removal composition can further be used to remove odors in places that are difficult to reach, such as, for instance, in jewelry clasps. It should be appreciated that the odor-removal composition may also be used to remove odors from other materials or items as the present invention is not intended to be limited in this manner. As used herein, the term “material” shall include any of the above items or any other objects or surfaces that may contain an odor.

### Salt Mixture

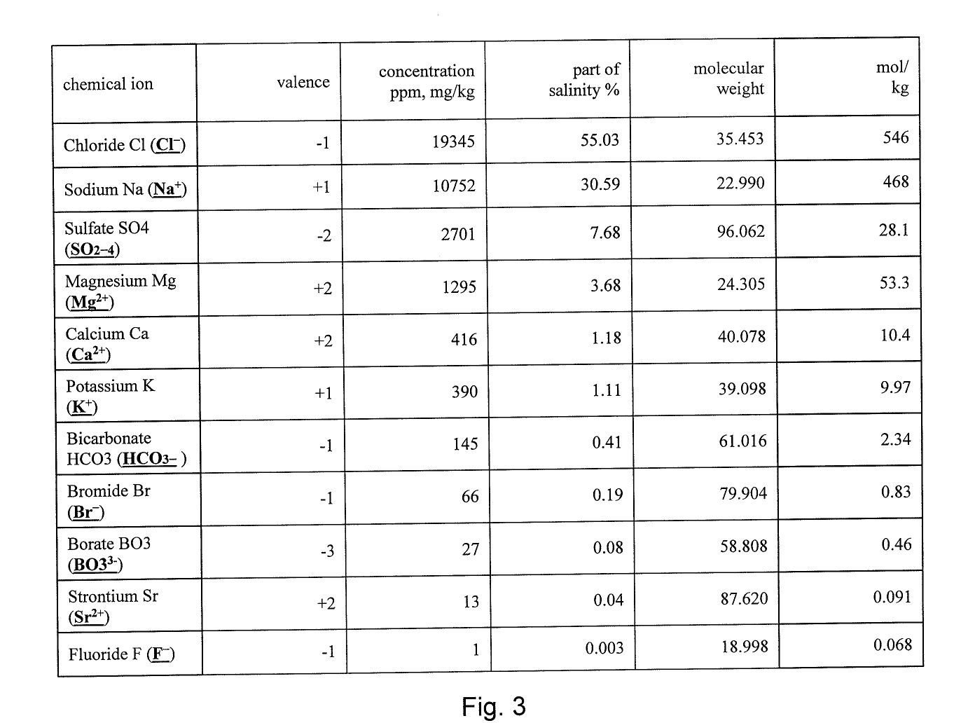

In an embodiment, the composition of the present invention may include a salt having at least one of a halogen and an alkaline in an effective amount sufficient to remove odors from the materials when applied thereto. The salt mixture may be selected from the group of ions including one of chloride, sodium, sulfate, magnesium, calcium, potassium, or a combination thereof. Of course, other ions may also be included in the salt mixture depending on the application. Examples of these other ions include, but are not limited to, bicarbonate, bromide, borate, strontium, fluoride, carbon or a combination thereof. FIG. 3 shows the chemical properties of the various ions that may be used in the salt mixture.

Chloride

In accordance with an embodiment of the present invention, the composition may include chloride. Chloride is a major anion, or negatively charged substance, in the blood and extracellular fluid. Blood and other body fluids have almost the same concentration of chloride ion as sea water. The balance of chloride ion (Cl⁻) is closely regulated by the body, as significant increases or decreases in chloride can have deleterious and even fatal consequences.

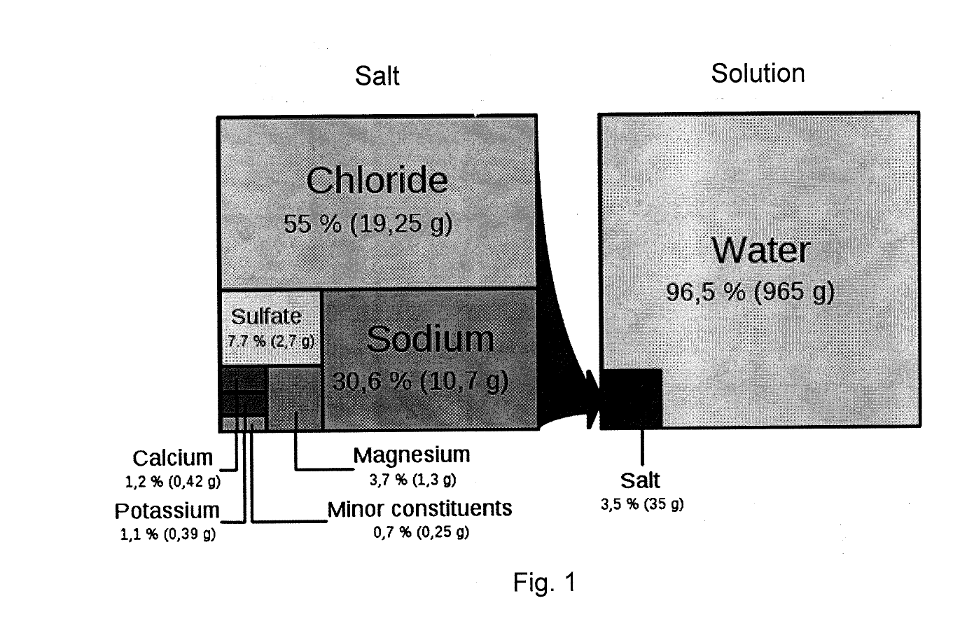

In an embodiment of the present invention, the composition may include chloride. In one embodiment, the composition may include from about 50% to about 60% of chloride by weight of the salt composition. As shown in FIG. 1, the composition may include about 19.25 grams of chloride. Of course, other dosages such as those above and below the noted ranges may also be suitable.

Sodium

In accordance with an embodiment of the present invention, the composition may include sodium. Sodium is an alkali metal and is soluble in water in nearly all of its compounds. A large amount of sodium is present in the Earth's oceans and other stagnant bodies of water. In these bodies it is mostly counterbalanced by the chloride ion, causing evaporated ocean water solids to consist mostly of sodium chloride, or common table salt. Sodium ion is also a component of many minerals.

In an embodiment of the present invention, the composition may include sodium. In one embodiment, the composition may include from about 25% to about 35% of sodium by weight of the salt composition. As shown in FIG. 1, the composition may include about 10.7 grams of sodium. Of course, other dosages such as those above and below the noted ranges may also be suitable.

Sulfate

In accordance with an embodiment of the present invention, the composition may include sulfate. Sulfate is a nonmetal that is derived from sulfur, which can be found as a bright yellow crystalline solid in it native form. In nature, it can be found as the pure element and as sulfide and sulfate minerals. It is an essential element for life and is found in two amino acids: cysteine and methionine. Its commercial uses are primarily in fertilizers, but it is also widely used in black gunpowder, matches, insecticides and fungicides.

In an embodiment of the present invention, the composition may include sulfate. In one embodiment, the composition may include from about 5% to about 10% of sulfate by weight of the salt composition. As shown in FIG. 1, the composition may include about 2.7 grams of sulfate. Of course, other dosages such as those above and below the noted ranges may also be suitable.

Magnesium

In accordance with an embodiment of the present invention, the composition may include magnesium. Magnesium (Mg) is an oil-soluble substance and is the fourth most abundant mineral in the body. Approximately 50% of total body magnesium is found in bone. The other half is found predominantly inside cells of body tissues and organs. Only 1% of magnesium is found in blood, but the body works very hard to keep blood levels of magnesium constant.

Magnesium is needed for more than 300 biochemical reactions in the body as it activates 76 percent of the enzymes in the body. Magnesium helps maintain normal muscle and nerve function, keeps heart rhythm steady, supports a healthy immune system, and keeps bones strong. A lack of magnesium, on the other hand, may inhibit nerve cell communication, which can lead to cell excitability. Magnesium also helps regulate blood sugar levels, promote normal blood pressure, and is known to be involved in energy metabolism and protein synthesis. Magnesium may further play a role in preventing and managing disorders such as hypertension, cardiovascular disease, and diabetes. Dietary magnesium is absorbed in the small intestines. Magnesium is excreted through the kidneys.

In an embodiment of the present invention, the composition may include magnesium. In one embodiment, the composition may include from about 1% to about 5% of magnesium by weight of the salt composition. As shown in FIG. 1, the composition may include about 1.3 grams of magnesium. Of course, other dosages such as those above and below the noted ranges may also be suitable.

Calcium

In an embodiment of the present invention, the salt mixture may include calcium. Calcium is a soft gray alkaline earth metal, and is the fifth most abundant element by mass in the Earth's crust and is also the fifth most abundant dissolved ion in seawater. Calcium is essential for living organisms, particularly in cell physiology, where movement of the calcium ion into and out of the cytoplasm functions as a signal for many cellular processes. Calcium is also the most abundant metal by mass in many animals. As such, it is a major material used in mineralization of bones and shells.

In an embodiment of the present invention, the composition may include calcium. In one embodiment, the composition may include from about 0.5% to about 2% of calcium by weight of the salt composition. As shown in FIG. 1, the composition may include about 0.42 grams of calcium. Of course, other dosages such as those above and below the noted ranges may also be suitable.

Potassium

In an embodiment of the present invention, the salt mixture may include potassium. Potassium is a soft silvery-white metallic alkali metal that oxidizes rapidly in air and is very reactive with water, generating sufficient heat to ignite the evolved hydrogen. Potassium can be found in nature only as ionic salt, and thus can be part of many minerals. Potassium ion is necessary for the function of all living cells, and is therefore present in all plant and animal tissues. It is found in especially high concentrations in plant cells, and in a mixed diet, it is most highly concentrated in fruits.

In an embodiment of the present invention, the composition may include potassium. In one embodiment, the composition may include from about 0.3% to about 1.5% of potassium by weight of the salt composition. As shown in FIG. 1, the composition may include about 0.39 grams of potassium. Of course, other dosages such as those above and below the noted ranges may also be suitable.

### Composition

The specific chemical properties of the odor-removing composition may vary according to specific applications; however, they may remain with substantially the same salinity. In the composition of the present invention, the salinity may be about 35 grams, or about 3.5% by weight of the solution. Salinity is a term that refers to the saltiness or dissolved salt content of the body of water. It is generally used to describe the levels of different salts such as sodium chloride, magnesium and calcium sulfates, and bicarbonates. Salinity is an ecological factor that may influence the types of organisms that live in a body of water. Moreover, density changes due to both salinity and temperature changes at the surface of the ocean produce changes in buoyancy, which cause the sinking and rising of water masses. Changes in the salinity of the oceans are further thought to contribute to global changes in carbon dioxide as more saline waters are less soluble to carbon dioxide. While the salinity of sea water varies worldwide, the relative abundance of the constituting ions can remain the same. Salinity is traditionally not described as a percent, but as parts per thousand, which is approximately grams of salt per kilogram of solution.

In accordance with an embodiment of the present invention, the composition may include water to deliver the composition. A variety of waters may be used, including, but not limited to tap water, recycled water, or natural waters such as rain and lake water. In certain embodiments, purified water (prepared by, e.g., distillation, reverse osmosis, or filtration) may be used because it contains fewer impurities that might reduce the effectiveness of the odor-removal composition. For example, certain impurities may cause algae to grow in the odor-removal composition. As a result, purified water may extend the life of the odor-removal composition. In certain circumstances, it may be desirable to seal the composition within a container away from ambient impurities. By sealing the composition, the shelf life of the odor-removal composition may be prolonged, and, in some circumstances, the shelf life may be virtually unlimited.

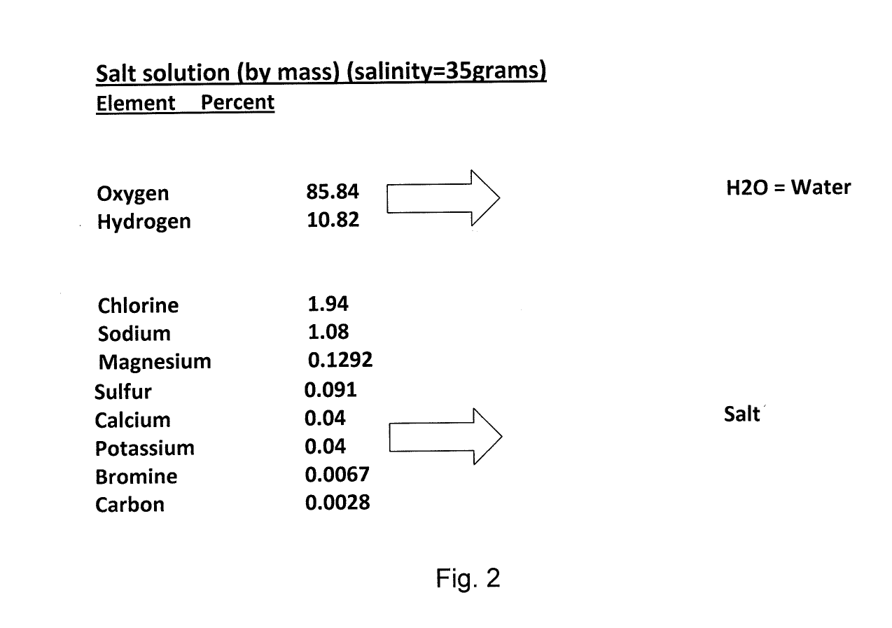

The composition for removing odors may, in one embodiment, include between about 90% to about 98% water by weight of the solution or about 900 grams to about 990 grams of water. As shown in FIG. 1, the composition may include about 965 grams of water, or about 96.5% water by weight of the solution. In another embodiment, the water may include oxygen and hydrogen. As shown in FIG. 2, the composition may include about 85.84% of oxygen and about 10.82% of hydrogen by weight of the solution. It should be appreciated that other ranges for water may also be suitable depending on the specific application or modality of use.

In an embodiment, the solution may include between about 2% and 10% of the salt mixture by weight of the solution or about 10 grams to about 100 grams of the salt mixture. As shown in FIG. 1, the solution may include about 3.5% of the salt mixture by weight of the solution. In another embodiment, the solution may include about 35 grams of salt mixture. It should be appreciated that other ranges for the salt mixture may also be suitable depending on the specific application or modality of use.

In an embodiment, the solution may include from about 1.5% to about 2.5% of chloride by weight of the solution, from about 0.75% to about 1.2% of sodium by weight of the solution, from about 0.07% to about 0.099% of sulfate by weight of the solution, include from about 0.10% to about 0.15% of magnesium by weight of the solution, from about 0.035% to about 0.055% of calcium by weight of the solution, and from about 0.035% to about 0.055% of potassium by weight of the solution. As shown in FIG. 2, the solution may include about 1.94% of chloride by weight of the solution, about 1.08% of sodium by weight of the solution, about 0.091% of sulfate by weight of the solution, about 0.1292% of magnesium by weight of the solution, about 0.04% of calcium by weight of the solution, and about 0.04% of potassium by weight of the solution.

In an embodiment, a solution of the composition can be created by combining, for instance, 1 large scoop of salt mixture with 1 gallon of water. One large scoop of the salt mixture is approximately 100 grams of salt mixture.

American washing machines by different manufactures use between about 13 and about 18 gallons of water for front loading and between about 30 and about 38 gallons of water for top loading washing machine per laundry cycle which includes rinsing. During the initial washing cycle when the detergent is released, between about 4 and about 5 gallons of water may be mixed with between about 3 and about 4 scoops of the salt mixture. This may allow washing for a full load of laundry. International washing machines may have the same volumes converted to liters.

For soaking, a solution of the composition can be created by combining 2 scoops with 1 to 2 gallons of water. Soaking may take approximately 15 to 20 minutes. It should be noted, however, that soaking may be longer or shorter if desired. The solution from the soaking goes directly into the washing machine along with the material. The solution may continue working in the washing machine and may not lose its effect even if combined with other detergent and clothing. In certain circumstances, the solution may enhances the wash and may brighten colors.

It should be appreciated that certain applications for the solution may require a more concentrated formula. In these situations, the composition may be adjusted to include more or fewer scoops.

### Applications

The odor-removal composition may be stored in various sized containers until it is used. Preferably, the odor removal composition is applied to odorous surfaces or materials at any time after exposure to the source of the odor. The composition of the present invention can remove even the toughest smell of body odor as well as odor in other materials, such as those mentioned above, by destroying the organisms that create the odor rather than by merely masking the stench.

The composition for removing odors can be used in a variety of applications or forms. It should be noted that the type of form used may depend on the type of material that has an odor. For instance, materials such as clothing, towels, bed linens or other materials that can be washed in a laundry unit may be treated using a different form than materials such as counter tops, tile floors, furniture or carpeting that are generally not washed in a laundry unit, or plastics or metals such as jewelry that are also generally not washed in a laundry unit. Moreover, humans and animals may also need a different form of treatment.

For example, the composition can be used as a granulate/powder formula that can be mixed with water. The composition in this form can be used for pre-soak and/or pre-wash cycles in cold and in warm water, or as a supplement to regular detergent at the start of the washing cycle. The granulate/powder form may dissolve with water and may not interfere with any product or brand. This form may further not affect the effectiveness of the washing detergent. The composition in this form may also be effective with basic hand wash application in a bucket or sink.

The composition may also be used as a liquid formula. The liquid formula, if desired, may be more concentrated than the granulate/powder formula. The composition in this form can be used for pre-soak and/or pre-wash cycles in cold and warm water, or mixed with any regular detergent at the start of the washing cycle. The liquid form may dissolve with water and may not interfere with any product or brand. This form may further not affect the effectiveness of the washing detergent. The composition in this form may also be effective with basic hand wash application in a bucket or sink.

Additionally, the composition may be used as prepacked pouches that can be added to and dissolved in water. This form is similar to the concentrated pouches used in dishwashing products in which the measurement is already pre-determined. The composition in this form can be used for pre-soak and/or pre-wash cycles in cold and warm water, or mixed with any regular detergent at the start of the washing cycle. The prepacked pouches may dissolve with water and may not interfere with any product or brand. This form may further not affect the effectiveness of the washing detergent.

The composition of the present invention can alternatively be used as dosing balls that can be added to water and which will dissolve in the water. Similar to the pouches, dosing balls may be made with pre-determined sizes that can dissolve in the water. The composition in this form can be used for pre-soak and/or pre-wash cycles in cold and warm water, or mixed with any regular detergent at the start of the washing cycle. The dosing balls may dissolve with water and may not interfere with any product or brand. This form may further not affect the effectiveness of the washing detergent.

In an embodiment, the odor-removal composition may be contained within a spray dispenser. The spray dispenser may be an aerosol dispenser that includes a container for holding the odor-removal composition and a valve member which will permit the odor-removal composition to be applied to the material containing the odorous material as a spray of fine, or finely divided, particles or droplets. The container may be sealed and pressurized by incorporating a gaseous propellant therein. Depression of an actuator coupled to the valve member may cause the odor-removal composition within the container to be dispensed. Commonly used propellants include gaseous hydrocarbons, such as isobutane, and mixed halogenated hydrocarbons. Because the use of hydrocarbon propellants is believed to be environmentally deleterious, alternative propellants such as compressed air, nitrogen, carbon dioxide, and inert gases may be used.

In another embodiment, the composition may be used as a pen for pre-treatment of materials, fabrics, or surfaces. The composition in this form may be used to treat specific areas or sections of materials that may require additional treatment to remove odors. For instance, the pen can be used to treat the underarms or other areas of clothing. Following the pretreatment, the material may undergo a wash cycle using regular detergent or a wash cycle using one of the form described above.

Moreover, the composition may be used as disinfecting wipes. The composition in this form may be used to treat specific areas or sections of materials that may otherwise not require subsequent treatment. In other words, the wipe may act to sufficiently remove any odors found on the material without the need to subject the material to subsequent treatment.

Additionally, the composition may be used as a sheet that can leave laundry smelling fresh and clean, similar to, for instance, a Bounce® fabric softener sheet. The composition in this form may be added to the drying cycle or can be placed on the material to sufficiently remove any odors found on the material.

Furthermore, the composition may be added through a sheet similar to a Bounce® sheet or a steam ball into washing machines and into dry cleaning cycles. The composition in this form, in one embodiment, may include a sheet of dried salt mixture. In another embodiment, the steam ball could have small pouches containing the salt mixture. When the composition is released through a steam cycle, the steam may interact with the salt mixture to activate the composition. The moist air containing the composition may then penetrate the fabric and may act to neutralize the sweat odor bacteria. In an embodiment, the composition in this form may not require the addition of water during washing cycles. In another embodiment, the composition in this form may use water during washing cycles.

In another embodiment, the composition may be applied to odor-containing material through a steam using, for instance, an iron or pressing machine. The composition in this form may use a sheet of dried salt mixture. The sheet may be applied to material, clothing or fabric during ironing or pressing. During the ironing or pressing of the material, clothing or fabric, the steam of the iron may interact with the sheet, allowing the moist air to penetrate the material, clothing or fabric and neutralize the sweat odor bacteria. In an embodiment, the composition in this form may not require the use of water.

It should be appreciated that two or more of the form mentioned above may be combined to aid in the removal of smells and odors while minimizing or reducing harm to the material or the color of the material. It should further be noted that the form mentioned above may be compatible and may be intermixed with any laundry process as well as any other cleaning liquids, powers, and detergents or other heavy duty cleaning solutions such as, for instance, Resolve®, Borax® and 409®. The form of the composition can be combined with any regular laundry detergent either independently by the consumer or sold to the detergent manufacturer and added by the detergent manufacturer directly into their product allowing them to enhance their product. Accordingly, the composition may not interfere with any other product or detergent. Using the composition may leave laundry smelling naturally clean and sterile. Natural fragrances, such as lavender or sandalwood, or essential oils, such as eucalyptus and mint, may be added to enhance a washed laundry smell.

Stand alone laundry odor removing formula can work with both cold and warm water washing cycles. As a cold water application, the composition can be used by individuals who do not have access to warm water, such as athletes who are competing in athletic competitions and campers who are camping. Utilization of the different forms of the composition may allow the consumer to use the composition at home or during travel.

In some embodiments, each of the different form of the composition can be used in the pre-soak laundering process in cold or in warm water. Use during the pre-soaking cycle may facilitate in neutralizing the odor before the regular washing cycle with regular detergent cleaning cycle commences. Use during the pre-soaking cycle may further aid in the removal of stains or in the enhancement of the fragrance.

The odor-removing composition of the present invention may also be biodegradable, environmentally safe, and energy saving. In an embodiment, the composition may be 100% bio-degradable or partially bio-degradable. In an another embodiment, the composition may be safe for consumers suffering from dermatological disorders.

### Experiments

In one experiment, unprocessed natural harvested sea salt from various countries (i.e., Portugal, Hawaii, Italy and Greece) was used to determine if there was any difference in using salt from various waters, such as, for example, the Atlantic Ocean, the Pacific Ocean, and the Mediterranean Ocean. In one instance, cold tap water was used in conjunction with purchased natural harvested sea salt from various regions of the world. The experiment showed no significant difference between the various salts and waters. In other words, the experiment showed that if a natural harvested sea salt, regardless of the country of origin, is combined any water in the correct proportion, the composition acts as an odor removing solution.

In a further experiment, regular fabrics like everyday products that interact with the body such as towels and sheets were pre-soaked with a solution of the composition. The experiment first included pre-soaking for anywhere from about 30 minutes to two hours in a bucket or sink of cold tap water with one large spoon of sea salt. Next, the pre-soaked towels and sheets were added to the washing machine along with regular detergent, such as, Oxy Clean®, ALL®, TIDE®, GAIN®, ARM & HAMMER®. The pre-soaked towels and sheets then underwent a regular washing cycle. The result was that the odor was gone and the towels and sheets had a clean fresh and almost sterile smell.

In another experiment, clothing underwent a regular washing cycle. The recommended manufactures dosage of detergent and added 3 scoops of the unprocessed sea salt formula was added to the cycle. The results showed that the clothing and synthetic fiber of sports clothing smelled clean and fresh. After using the clothing again for activity and sports activity, the clothing continued to smell fresh and the odor was gone.

In yet another experiment, the composition was used as a spray or wipe to clean sweat bacteria off of fitness equipment. In this experiment, a spray or wipe could be applied to various pieces of fitness equipment, such as, a tread mill, free weights, or any other piece of fitness equipment regardless of whether it operates electronically or not. In some instances, the spray or wipe could be used to reach sweat bacteria that is places within the pieces of fitness equipment that are difficult to reach with an ordinary towel. The spray or wipe could even be applied to the flooring surrounding the fitness equipment.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A composition comprising a salt having at least one of a halogen and an alkaline in an effective amount sufficient to remove odors from the materials when applied thereto.

2. A composition of claim 1 wherein the salt includes one of chloride, sodium, magnesium, calcium, potassium, or a combination thereof.

3. A composition of claim 1 further including sulfate.

4. A composition of claim 1 wherein the chloride is present from about 50% to about 60% by weight of the salt.

5. A composition of claim 1 wherein the sodium is present from about 25% to about 35% by weight of the salt.

6. A composition of claim 1 wherein the sulfate is present from about 5% to about 10% by weight of the salt.

7. A composition of claim 1 wherein the magnesium is present from about 1% to about 5% by weight of the salt.

8. A composition of claim 1 wherein the calcium is present from about 0.5% to about 2% by weight of the salt.

9. A composition of claim 1 wherein the potassium is present from about 0.3% to about 1.5% by weight of the salt.

10. A composition of claim 1 wherein the composition includes one of granulate, powder, liquid, prepacked pouches, dosing balls, spray, pen, disinfecting wipes, sheets, or a combination thereof.

11. A composition of claim 1 for use on natural fabrics, synthetic fabrics, carpeting, counter tops, plastics, metals, leather, tile floors, humans, pets, or a combination thereof.

12. A composition of claim 1 further including water to provide a solution.

13. A composition of claim 12 wherein the water is present from about 90% to about 98% by weight of the solution.

14. A composition of claim 12 wherein salinity of the solution is about 3.5% by weight of the solution.

15. A method of making a composition, comprising:
obtaining a salt having at least one of a halogen and an alkaline; and
processing the salt, such that it is suitable for administration to a material, and is in an effective amount sufficient to remove odors from materials when applied thereto.

16. A method of claim 15 wherein the salt includes one of chloride, sodium, sulfate, magnesium, calcium, potassium, or a combination thereof.

17. A method of claim 15 wherein processing includes adding the salt to water.

18. A method of claim 15 wherein processing includes one of granulate, powder, liquid, prepacked pouches, dosing balls, spray, pen, disinfecting wipes, sheets, or a combination thereof.

19. A method of claim 15 wherein administration includes presoaking the material.

20. A method of claim 15 wherein administering includes adding the composition to a wash cycle.
